(19) United States
(12) Patent Application Publication
(10) Pub. No.: US 2007/0059321 A1
(43) Pub. Date: Mar. 15, 2007

(54) Methods of reducing minimal inhibitory concentration of antibiotics and compositions resulting therefrom

(75) Inventors: Sissi Wachtel-Galor, Hong Kong SAR (CN); Iris Frances Forster Benzie, Hong Kong SAR (CN); Maureen Valerie Boost, Hong Kong SAR (CN)

Correspondence Address: BUCHANAN, INGERSOLL & ROONEY PC, POST OFFICE BOX 1404, ALEXANDRIA, VA, 22313-1404, US

(73) Assignee: The Hong Kong Polytechnic University, Hong Kong SAR (CN)

(21) Appl. No.: 11/224,291

(22) Filed: Sep. 13, 2005

(51) Int. Cl. A61K 36/06; A61K 38/14; A61K 38/16; A61K 31/43; A61K 31/545; A61K 31/407

(52) U.S. Cl. 424/195.16; 514/8; 514/192; 514/200; 514/210.09

(57) ABSTRACT

It is desirable to use lower doses of antibiotics in treating bacterial infections, as antibiotics are expensive and may come with undesirable side effects including kidney and/or liver damage. Moreover, using a high dose of antibiotics may lead to development of resistance in bacteria to the antibiotics themselves. More importantly, increasing the dosage may not be able to increase the amount of antibiotics entering the body. Therefore, it may be desirable to devise methods to lower the minimal inhibitory concentration of antibiotics. This invention provides a method to reduce the minimal inhibitory concentration of antibiotics by adding Ganoderma lucidum, or commonly know as Lingzhi, extracts, and therefore, lower does of antibiotics may be required.

### FIELD OF THE INVENTION

This invention relates to methods and compositions of reducing minimal inhibitory concentration of antibiotics

### BACKGROUND OF THE INVENTION

Misuse of antibiotics is now a major issue in the development of resistance in bacteria to the antibiotics themselves. One particular and important problem is the increase in prevalence of Methicillin Resistant Staphylococcus aureus (MRSA), which causes serious, and often fatal, infections in hospitalized patients. MRSA is resistant to penicillin and methicillin and usually responds to only two currently available antibiotics, vancomycin and linezolid. However, some strains of MRSA already exhibit resistance to these two antibiotics. If the minimal inhibitory concentration (MIC) of an antibiotic can be reduced, there may be a chance to restore the therapeutic efficacy of antibiotics that have ceased to be therapeutically useful because of the development of resistance in microorganisms. Such resistance is reflected in the rise of MIC to be above the achievable therapeutic range. As generally noted, increasing the dosage may not increase the therapeutic efficacy of antibiotics, as the amount of antibiotics entering the body may not increase after a certain threshold value. Therefore, decreasing the MIC may represent an important indication that there is at least a chance to restore the once lost therapeutic efficacy of antibiotics.

Further, antibiotic treatment is expensive, and may come with undesirable side effects including kidney and/or liver damage. High doses of certain antibiotics may also cause serious side effects, for example nerve damage leading to deafness. On the other hand, high doses are needed if the bacteria are resistant to the antibiotic being used.

Therefore, there is a need to develop a method and composition so that the efficacy of antibiotics may be improved, and/or lower doses of antibiotics can be used to treat bacterial infections.

### OBJECTS OF THE INVENTION

Therefore, it is an object of this invention to provide a method and/or composition with reduced MIC, so that at least lower doses of antibiotics can be used to treat bacterial infections so that at least one or more of the problems as set forth in the prior art may be resolved. As a minimum, it is an object of this invention to provide the public with a useful choice.

### SUMMARY OF THE INVENTION

Accordingly, this invention provides a method of reducing minimal inhibitory concentration of an antibiotic by mixing the antibiotic with an extract of Ganoderma lucidum.

Preferably, the antibiotic belongs to beta lactam group, more preferably selected from the group consisting of penicillin, methicillin, ampicillin, piperacillin, first, second, and third generation cephalosporins, and carbapenems. Alternatively, the antibiotic belongs to glycopeptide group, which may be selected from the group consisting of vancomycin and teicoplanin.

Preferably, extract of Ganoderma lucidum is in an amount of at least 1% w/v.

It is another aspect of this invention to provide a composition including an antibiotic and an extract of Ganoderma lucidum.

It is yet another aspect of this invention to provide a method of manufacturing a medicament by combining an antibiotic with an extract of Ganoderma lucidum.

### DETAILED DESCRIPTION OF THE PREFERRED EMBODIMENT

Objects, features, and aspects of the present invention are disclosed in or are obvious from the following description. It is to be understood by one of ordinary skilled in the art that the present discussion is a description of exemplary embodiments only, and is not intended as limiting the broader aspects of the present invention, which broader aspects are embodied in the exemplary constructions.

According to this invention, it is surprisingly found that extracts of the woody mushroom Ganoderma lucidum (also known as “Lingzhi” and “Reishi”) are capable of lowering the minimal inhibitory concentration (MIC) of antibiotics. MIC is defined as the lowest concentration that results in inhibition of visible growth of the organism. The G. lucidum extracts may include, but not limited to hot water extract; water-ethanol (polysaccharide-rich) extracts; methanol-chloroform (triterpene-rich) extracts of the fruiting body and/or spores of G. lucidum.

It is also found that the amount of the extracts added to the antibiotics can be very small to be effective, as small as 1% w/v. Obviously amount of the extracts cannot be 100%, otherwise no antibiotics are present in the formulation. However, there is no report of Lingzhi toxicity in the literature. Therefore, it appears to be safe even if high doses of the Lingzhi extracts are used, for example 30 g per day.

Many antibiotics can be used with the Lingzhi extracts, including the beta lactam group and glycopeptide group of antibiotics. Examples of the beta lactam group of antibiotics include penicillin, methicillin, ampicillin, piperacillin, cephalosporins, carbapenems. Representatives of the glycopeptide group of antibiotics may include vancomycin and teicoplanin.

Various Lingzhi extracts may be used, including hot water extract, alcohol extract (polysaccharide rich extract), or even commercially available extracts may be used. Details will be described in the following section.

### EXAMPLES

Extraction Procedure Preparation

The fruit bodies of the dried mushroom were washed under running tap water followed by distilled water. The fruit bodies were then put on the bench at room temperature (˜20° C.) until dry (around 2 days). After drying, the fruit bodies were cut into small pieces and extracted.

Extract 1: Mushroom Hot Water (MHW)

Dried fruit bodies were extracted with distilled water at 100° C. (in water bath) for 3 hours, and mixed every 15 min. After extraction, the samples were filtered (Whatman filter paper #2) and the filtrate was freeze-dried or lyophilized (Labconco Corp., Kansas City, Mo., USA). This extract was called Mushroom Hot Water (MHW).

Extract 2: Mushroom Polysaccharide Rich (MPR)

Fruit bodies were extracted with distilled water at 100° C. for 3 hours (as for hot water extract above). After extraction, the samples were filtered and the filtrate was concentrated by lyophilization. Absolute ethanol was then added to the concentrate (80% saturation) and the mixture was allowed to stand overnight at 4° C. The precipitate was collected by centrifugation (Sorvall RC-2B, Kendro Laboratory Products, Ashevillence, N.C., USA). The precipitant was re-dissolved in distilled water and dialyzed at 4° C. overnight against distilled water (10K cut-off membrane). The dialyzed material was then lyophilized.

Extract 3: Commercial Hot Water (CHW)

Commercially available capsules containing an extract of G. lucidum were also tested. For the samples used in this study, a Certificate of Analysis was provided. These commercial extracts are sold as ‘over the counter’ dietary supplements in pharmacies and drug stores in Hong Kong as of now (2005).

Preparations

Water preparations of fruit bodies or commercial extracts were prepared. For preparations of water extracts, 1% w/v commercial extract (CHW), mushroom's fruit bodies hot-water extract (MHW) and mushroom's fruit bodies polysaccharide-rich extract (MPR) were weighed and dissolved in boiling water. The mixtures were then vortexed and then roller mixed for the next 30 minutes. The suspensions were filtered and the filtrates collected for further investigation.

Determination of Minimal Inhibitory Concentration

Antimicrobial susceptibility testing was performed by broth assay followed standard laboratory procedures (the National Committee for Clinical Laboratory Standards (NCCLS) protocol 2000). The MIC in this assay was defined as the lowest concentration of the antibiotic under test that inhibited the visible growth of the microorganism under study.

There were eight strains of Staphylococcus aureus tested. All were from clinical isolates as they represent strains actually present in the community and so of clinical relevance. Three strains were sensitive to methicillin (MSSA), and five strains were resistant to methicillin (MRSA).

The MIC for each of the eight strains was determined for penicillin G and for vancomycin using dilutions made from the stock preparations purchased (both from Sigma). The effect of G. lucidum extracts (MHW, MPR and CHW) on the MICs was investigated by adding each type of extract (individually and at 1% w/v final concentration in the test mixture in each case) alone and in combination with each dilution of each antibiotic tested and against all 5 strains of Staphylococcus aureus. The details are illustrated in the following paragraphs.

The antimicrobial susceptibility test was performed in a 96-well microtitre plate and following standard National Committee for Clinical Laboratory Standards (NCCLS) protocol (NCCLS, 2000). In this assay, two-fold dilution of the examined agent is performed sequentially along the rows. Minimal inhibitory concentration (MIC) was determined by the microdilution method in Mueller-Hinton broth (Oxoid, Hampshire, UK), according to the standards of the NCCLS (2000). The MIC was defined as the lowest concentration that results in inhibition of visible growth of the organism.

The MIC for each of the strains was determined for penicillin G (Sigma) and vancomycin (Sigma) by broth microdilution, using dilutions from a stock concentration of each antibiotic of 4096 μg/ml (NCCLS, 2000).

The different extracts were: mushroom's fruit body hot water extract (MHW), polysaccharide-rich extract (MPR), and a commercial extract (CHW)-preparations of the extracts, source of the fruit bodies and commercial extracts are described in the section following the results. The G. lucidum preparations were adjusted to pH 7. The final concentration of the G. lucidum extracts in the microplates was 1% w/v. The antibiotics and G. lucidum were both filtered before use.

Each bacterial strain was plated overnight on Mueller-Hinton agar and then transferred to a broth 3 hours before the assay was performed. 5 μl of bacterial suspension adjusted to the turbidity of the 0.5 McFarland standard was added to each well, to yield a final inoculum of ˜4×10⁴ CFU/well. Plates were covered and incubated at 37° C. Microplates were examined 18 hours later and determined for MIC. The MIC was the lowest concentration of antimicrobial agent that yielded no growth by visual reading after incubation.

Samples of each strain of bacteria were incubated with an antibiotic alone, G. lucidum alone or a combination of the two agents. The first experiments were to find the MIC concentrations of the antibiotics for the different strains examined. The next set of experiments tested the effect of each of the three types of G. lucidum alone and in combination with each of the antibiotics.

The Fractional Inhibitory Concentration (FIC) was also calculated. The FIC is defined below and is an indication of whether a combined treatment is synergistic, additive, has no effect or is antagonistic.

$${FIC}_{( A )} = \frac{MIC ( drug A in the presence of B )}{MIN ( A alone )}$$

As generally accepted in the art:

FIC less than or equal to 0.5 indicates a synergistic effect;

FIC between 0.5-1.0 indicates an additive effect;

FIC between 1.0-2.0 indicates no effect; and

FIC greater than or equal to 2.0 indicates an antagonistic effect.

Results

None of the three Linghzi extracts showed inhibition of the different strains at concentrations up to 1% (final concentration). MIC concentrations of the different strains to penicillin ranged from 8 to 1024 μg/ml and, to vancomycin, between 0.5 to 1 μg/ml (Table 1). The fractional inhibitory concentration (FIC) results are summarised in table 2. As can be seen, G. lucidum showed synergistic or additive effects with both penicillin and vancomycin against the tested strains of MSSA and MRSA.

TABLE 1
MICs of G. lucidum and tested antibiotics

| Bacterial | Pen | Pen + MHW | Pen + CHW | Pen + MPR | Van | Van + MHW | Van + CHW | Van + MPR |
|---|---|---|---|---|---|---|---|---|
| strain | MIC | MIC | MIC | MIC | MIC | M1C | MIC | MIC |
| MSSA1 | 64 | 16 | 8 | 8 | 0.5 | 0.5 | 0.5 | 0.25 |
| MSSA2 | 32 | 16 | 8 | 4 | 0.5 | 0.5 | 0.5 | 0.5 |
| MSSA3 | 8 | 4 | 2 | 1 | 0.5 | 0.25 | 0.125 | 0.125 |
| MRSA1 | 512 | 128 | 64 | 64 | 0.5 | 0.125 | 0.125 | 0.125 |
| MRSA2 | 256 | 32 | 32 | 16 | 1 | 0.5 | 0.125 | 0.5 |
| MRSA3 | 1024 | 256 | 128 | 128 | 1 | 0.5 | 0.5 | 0.5 |
| MRSA4 | 256 | 32 | 16 | 8 | 0.5 | 0.5 | 0.25 | 0.25 |
| MRSA5 | 512 | 64 | 32 | 64 | 0.5 | 0.25 | 0.125 | 0.125 |

(Pen-penicillin; Van = vancomycin; MHW = mushroom hot water extract; CHW = commercial extract; MPR = mushroom polysaccharide-rich extract)

TABLE 2
FICs of G. lucidum and tested antibiotics

| Bacterial | Pen + MHW | Pen + CHW | Pen + MPR | Van + MHW | Van + CHW | Van + MPR |
|---|---|---|---|---|---|---|
| strain | FIC | FIC | FIC | FIC | FIC | FIC |
| MSSA1 | 0.25 | 0.125 | 0.125 | 1.0 | 1.0 | 0.5 |
| MSSA2 | 0.5 | 0.25 | 0.125 | 1.0 | 1.0 | 1.0 |
| MSSA3 | 0.5 | 0.25 | 0.125 | 0.5 | 0.25 | 0.25 |
| MRSA1 | 0.25 | 0.125 | 0.125 | 0.25 | 0.25 | 0.25 |
| MRSA2 | 0.125 | 0.125 | 0.0625 | 0.5 | 0.125 | 0.5 |
| MRSA3 | 0.25 | 0.125 | 0.125 | 0.5 | 0.5 | 0.5 |
| MRSA4 | 0.125 | 0.0625 | 0.03125 | 1.0 | 0.5 | 0.5 |
| MRSA5 | 0.125 | 0.0625 | 0.125 | 0.5 | 0.25 | 0.25 |

The results of the experiments show that G. lucidum or Lingzhi extracts are able to lower the minimal inhibitory concentration of different antibiotics. Therefore, a lower amount of dosage rate of antibiotics may be used to treat infections in conjunction with Lingzhi extracts.

None of the G. lucidum extracts tested showed a direct effect (at 1% w/v) in regard to inhibition of growth of any of the strain tested.

There was a significant MIC lowering effect seen with all strains for penicillin in the presence of G. lucidum extract tested. In some cases the MIC was reduced back into the therapeutic range for penicillin (known in the art as 4 ug/ml). In some cases the susceptibility of the organism to the antibiotic was increased up to 16 fold or more.

For vancomycin, evidence of increased efficacy can also be observed in the presence of each G. lucidum extract.

The FIC results as presented in Table 2 show evidence of additive or synergistic effects between G. lucidum and penicillin, and between G. lucidum and vancomycin against the tested strain of MSSA and MRSA.

### CONCLUSIONS AND SUMMARY

Results presented show that G. lucidum extracts are able to lower the Minimal Inhibitory Concentration (MIC) of different antibiotics against clinically relevant strains of pathogenic bacterial including, importantly, methicillin resistant Staphylococcus aureus (MRSA). This means that the efficacy of these antibiotics is improved in the presence of G. lucidum extracts. In some cases the MIC was reduced back to the achievable therapeutic range.

Results indicate that well tolerated, established and inexpensive antibiotics (for example penicillin) that are often no longer used because of antibiotic resistance to their action can be restored to have therapeutic efficacy when used in a combination with an extract of G. lucidum.

Results also indicates that, in combination with G. lucidum, the same therapeutic effect may be achieved with a lower dose. In another words, same dose of an antibiotic, for example vancomycin, may achieve a significantly greater therapeutic effect.

While the preferred embodiment of the present invention has been described in detail by the examples, it is apparent that modifications and adaptations of the present invention will occur to those skilled in the art. Furthermore, the embodiments of the present invention shall not be interpreted to be restricted by the examples or figures only. It is to be expressly understood, however, that such modifications and adaptations are within the scope of the present invention, as set forth in the following claims. For instance, features illustrated or described as part of one embodiment can be used on another embodiment to yield a still further embodiment. Thus, it is intended that the present invention cover such modifications and variations as come within the scope of the claims and their equivalents.

1. A method of reducing minimal inhibitory concentration of an antibiotic including the step of mixing the antibiotic with an extract of Ganoderma lucidum.

2. The method of claim 1, wherein the antibiotic belongs to beta lactam group.

3. The method of claim 2, wherein the antibiotic is selected from the group consisting of penicillin, methicillin, ampicillin, piperacillin, first, second, and third generation cephalosporins, and carbapenems.

4. The method of claim 1, wherein the antibiotic belongs to glycopeptide group.

5. The method of claim 4, wherein the antibiotic is selected from the group consisting of vancomycin and teicoplanin.

6. The method of claim 1, wherein the extract of Ganoderma lucidum is in an amount of at least 1% w/v.

7. A composition including:
an antibiotic; and
an extract of Ganoderma lucidum.

8. The composition of claim 7, wherein the antibiotic belongs to beta lactam group.

9. The composition of claim 8, wherein the antibiotic is selected from the group consisting of penicillin, methicillin, ampicillin, piperacillin, first, second, and third generation cephalosporins, and carbapenems.

10. The composition of claim 7, wherein the antibiotic belongs to glycopeptide group.

11. The composition of claim 10, wherein the antibiotic is selected from the group consisting of vancomycin and teicoplanin.

12. The composition of claim 7, wherein the extract of Ganoderma lucidum is in an amount of at least 1% w/v.

13. A method of manufacturing a medicament comprising the steps of combining an antibiotic with an extract of Ganoderma lucidum.

14. The method of claim 13, wherein the antibiotic belongs to beta lactam group.

15. The method of claim 14, wherein the antibiotic is selected from the group consisting of penicillin, methicillin, ampicillin, piperacillin, first, second, and third generation cephalosporins, and carbapenems.

16. The method of claim 13, wherein the antibiotic belongs to glycopeptide group.

17. The method of claim 16, wherein the antibiotic is selected from the group consisting of vancomycin and teicoplanin.

18. The method of claim 13, wherein the extract of Ganoderma lucidum is in an amount of at least 1% w/v.
